(19) United States
(12) Patent Application Publication
(10) Pub. No.: US 2022/0280077 A1
(43) Pub. Date: Sep. 8, 2022

(54) GLUCOSE ELECTROLYSIS METHOD AND APPARATUS

(71) Applicant: Douglas Stockton, Tucson, AZ (US)

(72) Inventor: Douglas Stockton, Tucson, AZ (US)

(21) Appl. No.: 17/317,319

(22) Filed: May 11, 2021

Related U.S. Application Data
(60) Provisional application No. 63/158,156, filed on Mar. 8, 2021.

(51) Int. Cl. A61B 5/1473; C25B 3/25; C25B 9/23

(52) U.S. Cl. CPC A61B 5/1473; C25B 3/25; C25B 9/23

(57) ABSTRACT

A glucose electrolysis apparatus for breaking down glucose and reducing osmolality of the blood includes a catheter having an anode located at a distal end of the catheter. A cathode is connected to the anode by a reduction wire located within the catheter. A mesh covers the anode to exclude molecules from the catheter. A power source is connected to the reduction wire to drive a reaction forward on the anode surface.

### CLAIM OF PRIORITY

This application claims priority from Provisional Application Ser. No. 63/158,156 filed on Mar. 8, 2021, the entirety of which is hereby incorporated by reference.

### BACKGROUND OF THE DISCLOSURE

This disclosure relates to treatment of diabetes. More particularly, it relates to a glucose electrolysis method and apparatus for breaking down blood glucose concentrations and reducing osmolality of the blood. Diabetes mellitus is a condition that affects approximately 10.5% of the United States population with rising prevalence. Diabetes mellitus can be separated into two different types: type 1 and type 2. Type 1 diabetes mellitus is a metabolic disorder caused by autoimmune destruction of pancreatic beta cells which produce insulin. Type 2 diabetes mellitus is a metabolic disorder caused by insulin resistance of organs throughout the human body. Insulin is a hormone produced by the pancreatic beta cells which allows the body to uptake glucose and other sugars from the bloodstream. Glucose needs to be taken into cells and out of the bloodstream to be metabolized and utilized. Approximately 95% of cases of diabetes mellitus fall into the type 2 category. Some major risk factors for developing type 2 diabetes mellitus are obesity, increased age, family history, certain ethnicities, hypertension, and hypercholesterolemia. Insulin resistance leads to hyperglycemia (high concentrations of glucose in the blood). Hyperglycemia alone can cause symptoms of polyuria (excessive urination) which results in polydipsia (excessive thirst). Long term effects of diabetes mellitus are atherosclerosis (plaque build-up) of arteries, kidney disease and failure, peripheral neuropathy (loss of sensation in fingers and toes), cataracts, blindness, male impotence, and limb amputation.

Excessively high levels of hyperglycemia (>600 mg/dL) can result in a hyperosmotic hyperglycemic nonketotic state (HHNK) which often presents with symptoms of polyuria, polydipsia, fever, dehydration, hyperosmolar blood, and confusion. Inciting factors include infection, medications that impair glucose control, and lack of proper use of glucose controlling medications. The polyuria and dehydration results in hyperosmolality (increased concentration) of the blood. Hyperosmolality will draw water out of the brain, resulting in seizures, coma, and death in up to 20% of patients in HHNK. Current treatment regimens for HHNK involve intravenous saline, insulin, and electrolyte correction, typically for hypokalemia (low concentration of potassium in the blood).

There is a need for an apparatus and method for breaking down blood glucose to decrease osmolality of the blood.

### SUMMARY OF THE DISCLOSURE

The present disclosure is directed to a catheter. More particularly, it is directed to an electrode structure provided on an end of the catheter. In a patient in HHNK or with high blood glucose concentrations, the catheter would be inserted into the bloodstream, such as through the neck, leg, etc., and the electrode would run a current through the blood to break down glucose into basic components, decreasing the osmolality of the blood. This could be done alone, or, more preferably, in conjunction with current treatment regimens of intravenous saline, insulin, and electrolyte correction. Also provided are methods of producing the catheter with the electrode placed on the head.

The catheter of the present disclosure involves direct glucose oxidation on a metal cathode. A mesh covers the metal cathode. The mesh preferably would be porous for molecules around the size of glucose (approximately 1 nm) to pass through

Recent research has been conducted on accurately quantifying glucose concentration by voltammetry with the use of enzyme mediated electrodes, but there have been efforts to find a direct metal catalyst which would decrease the cost of glucose concentration determination.

However, the goal in the present disclosure is not to determine glucose concentration, rather to simply oxidize the glucose into smaller compounds to decrease a patient's blood glucose levels. A great deal of recent research on glucose oxidation has been done on creating clean energy, and most of it involves only the first step of glucose oxidation, which creates gluconolactone. However, that still leaves a potentially toxic substance in the body. A recent study has been performed to create an electrode catalyst that would completely oxidize glucose into carbon dioxide and water as this would provide more energy per glucose molecule, and thus be much more energy beneficial in clean energy. This is done through an “enzyme cascade” which provides all the steps in glucose oxidation (see “Enzymatic Biofuel Cell for Oxidation of Glucose to CO₂”, Shuai Xu and Shelley D. Minteer (ACS Catal. 2012, 2, 91-94), which is incorporated by reference herein).

In accordance with a preferred embodiment of the disclosure, a glucose electrolysis apparatus has a catheter having an anode located at a distal end of the catheter; and a cathode connected to the anode both by a wire within the catheter and an aqueous medium. A mesh covers the anode to exclude larger molecules in the bloodstream from the catheter. A power source is connected to the wire to drive an oxidation reaction forward on the anode surface.

In accordance with another embodiment of the disclosure, a method of oxidation of glucose includes the steps of: providing a catheter, providing an anode at a distal end of the catheter, connecting a cathode to the anode by a reduction wire located within the catheter, covering the anode with a mesh cover to exclude molecules from entering the catheter, connecting a power source to the reduction wire to drive an oxidation reaction forward on the anode surface, and inserting the catheter into the bloodstream.

One aspect of the present disclosure is to use a cathode to completely oxidize glucose to carbon dioxide and water.

Another aspect of the present disclosure would be to place the catheter in the bloodstream of a patient. This can be accomplished in numerous different ways.

Still another aspect of the disclosure would be to access the femoral vein, subclavian vein, or internal jugular vein. This method is easily accomplished by the Seldinger technique common to medical providers gaining access to a blood vessel. The device would sit in the large vessels of the body and break down glucose.

Another aspect of the disclosure is that ultrasound and/or Xray could be used to assist in placement of the catheter.

Still other aspects of the disclosure will be apparent upon a reading and understanding of the following detailed description.

### BRIEF DESCRIPTION OF THE DRAWINGS

The disclosure is best understood by utilizing the accompanied drawings within the detailed description.

FIG. 1 is a schematic diagram of the cathode in a bloodstream and a power supply in accordance with a preferred embodiment of the disclosure.

FIG. 2 is a top plan view of the catheter and power supply in accordance with a preferred embodiment of the disclosure.

FIG. 3 is an enlarged perspective view of an electrode at an end of the catheter of FIG. 2.

FIG. 4 is an enlarged perspective view of a power supply attached to the catheter of the present disclosure.

FIG. 5 is an enlarged side elevational cross section view of the electrode at the end of the catheter of the present disclosure.

FIG. 6 is an enlarged side elevational view of the electrode at the end of the catheter in accordance with the disclosure.

FIG. 7A is a perspective view of the end of the catheter of the present disclosure.

FIG. 7B is a perspective view in cross section of the end of the catheter of the present disclosure.

### DETAILED DESCRIPTION OF THE DISCLOSURE

Before any specific details are described, it should be noted that this disclosure is not limited to the applications described, as applications may vary. It should be understood that the terminology used is for describing particular embodiments only, and the range of applications for this device should not be limited by the appended claims.

All terms not distinctly defined have the same meaning as used by a member of ordinary skill in the field which this invention may be used in.

The following definitions are used for clarification:

An “electrode” is a conducting surface where a reaction of electron gain or loss takes place.

A “working electrode” is the electrode where the reaction of interest takes place.

A “counter electrode” is an electrode used in conjunction with a working electrode to provide a circuit for which electrons to flow.

“Electrolysis” is the process by which a molecule is broken down by gain or loss of electrons.

An “electrolytic cell” uses electrical energy to drive a non-spontaneous redox reaction forward.

A “galvanic cell” uses a spontaneous redox reaction to drive a circuit forward.

“Oxidation” is the loss of electrons by a molecule.

“Reduction” is the gain of electrons by a molecule.

An “anode” is an electrode where electrons leave a substance by oxidation to flow towards a cathode.

A “cathode” is an electrode where electrons leave a circuit to be gained by a substance by reduction.

Gibbs free energy is a thermodynamic term used to define the spontaneous nature of a chemical reaction by incorporating both enthalpy and entropy into its use. A negative Gibbs free energy reaction is thought to be spontaneous meaning that it will happen under normal conditions. A positive Gibbs free energy reaction is thought to be nonspontaneous, meaning that the reaction will not occur under normal conditions. Gibbs free energy only defines if a reaction will occur or not, but it does not reflect on the kinetics or timing of a reaction.

A “catheter” is a flexible tube that is inserted into the body through a small hole, often with the purpose of removing fluids or other materials from the body.

A “sheath” is a tube that is inserted partially into the body to help a medical professional insert catheters and wires into and out of the body.

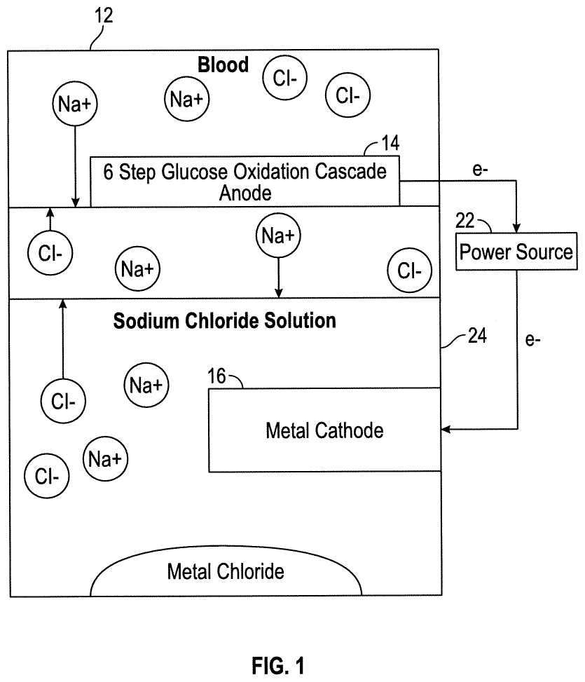

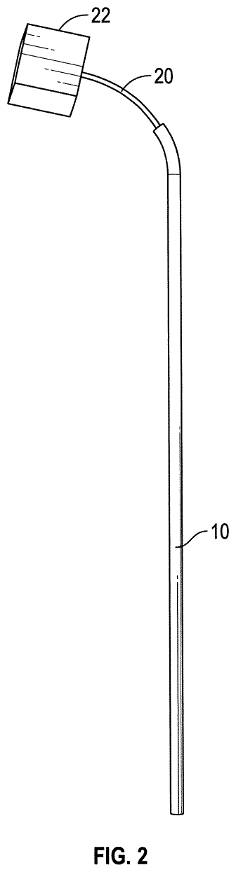

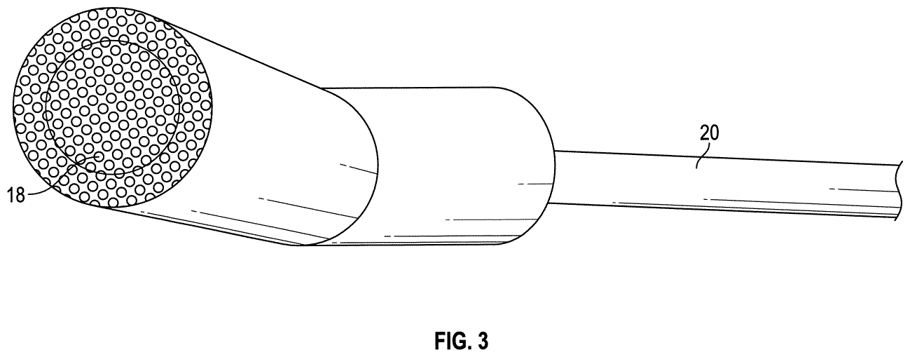

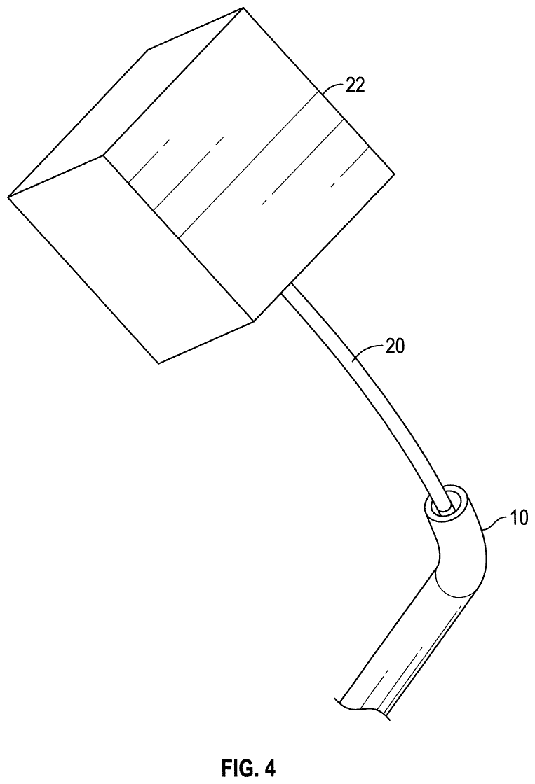

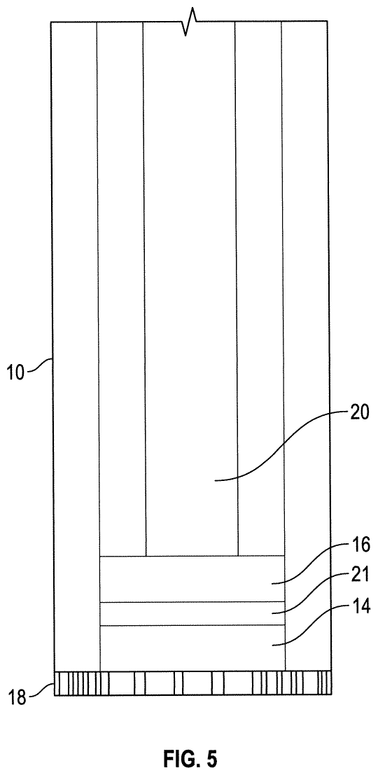

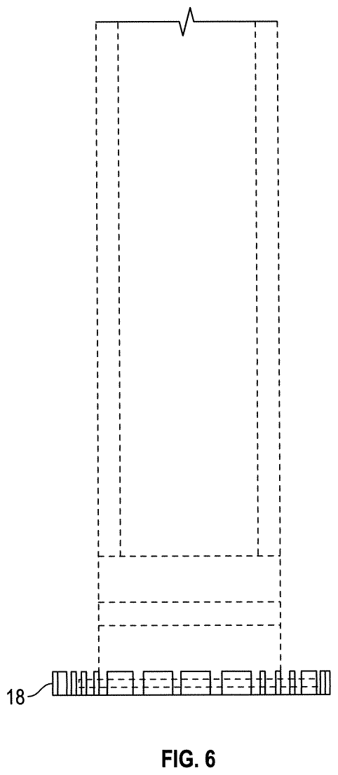

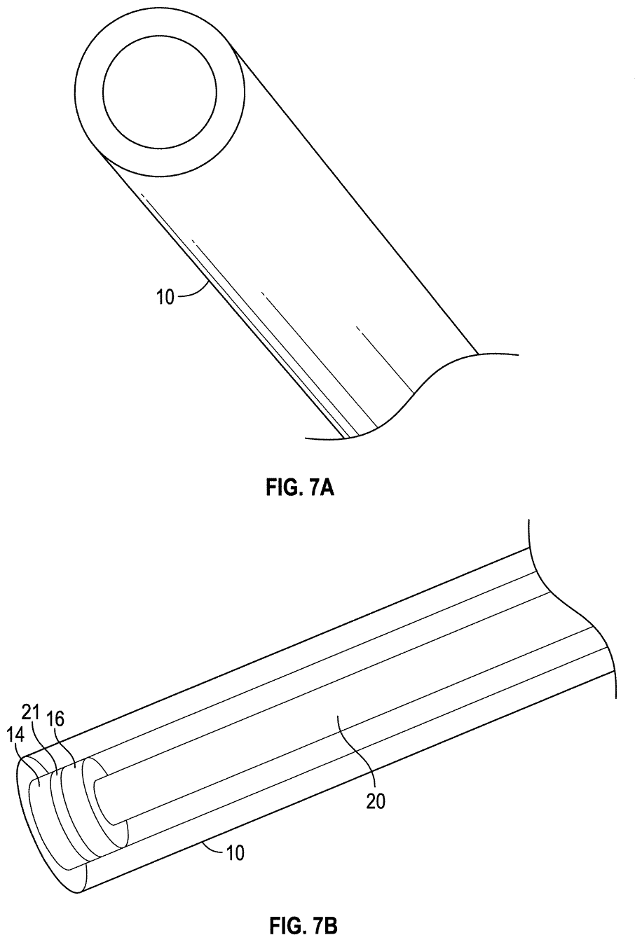

The device of the present disclosure includes a catheter 10 on the outside of the body. Referring to FIGS. 2, 3, 4, 7A and 7B, catheters 10 are elongated flexible cylinders or tubes that are inserted through a sheath into the bloodstream 12 (FIG. 1). An anode 14 would be placed at the distal end of the catheter which would be placed into the bloodstream. The anode 14 would cover most of the surface area of the cathode tip. The anode would be connected to a cathode 16 by a metal wire inside of the catheter 10 which would provide a means for a circuit to be completed. The cathode would not necessarily have to be encased inside the catheter; rather, it could be placed outside of the catheter. The anode 14 would preferably have a porous mesh 18 over an outer surface to provide a means for simple size exclusion selection of molecules. The rest of the catheter would be a flexible cylinder with a wire 20 running through it to drive the reaction. The proximal end of the catheter would be connected to an energy source 22 to drive the reaction forward.

The metal anode 14 would preferably have a bio enzyme cascade. This would involve multiple enzymes on the surface of the metal anode to drive the reaction forward as well as completely oxidize glucose to carbon dioxide and water. Currently available is a paper electrode anode coated with a polymer necessary to hold the enzymes on the surface. The bio enzyme cascade is completed with six steps. The enzymes used are pyrroloquinoline quinone (PQQ) dependent glucose dehydrogenase, PQQ-dependent 2-gluconate dehydrogenase, aldolase, PQQ-dependent alcohol dehydrogenase, PQQ-dependent aldehyde dehydrogenase, and oxalate oxidase. The anode 14 would come into direct contact with the bloodstream 12 of the user.

The cathode 16 used could be any conventional cathode material. For example, some common materials are silver/silver chloride, platinum, and lithium based metal combinations. The cathode material does not have to have a specific material type; rather, it has to be capable of having a reduction reaction occur on its surface to drive the chemical reaction forward. The cathode would also not come into contact with any part of the human body. Rather, it would be placed inside the catheter 10 or at or near the proximal end of the catheter which is never advanced into the body.

The anode 14 and cathode 16 would have to be connected with a material or conductor that would allow the circuit to be completed. There are myriad materials available to the common user; typically, the best material has low electrical resistance to increase conductance, and would not react at the potential ranges used for the electrical circuit. The conductor could be, but is not limited to, copper, aluminum, silver, steel, iron, gold, a combination or conductors, a synthetic polymer substance, or any other conductive material known to the common user. The conductor would be insulated with a dielectric material to decrease loss of electrical flow. The dielectric material could be, but is not limited to, flexible plastic or any other dielectric material known to the common user.

The anode 14 and cathode 16 would be connected to a power source 22 to drive the reaction forward through the blood stream. This reaction is a spontaneous redox reaction, so it does not need a power source for it to happen. However, the reaction on its own is relatively slow, so a power source is preferably used to speed up the reaction. The power source 22 could be, but is not limited to, a battery, an electrical outlet, or any other conventional source of power.

The anode 14 and cathode 16 would need to be connected to each other with a medium 21 for which ions can transfer to keep solutions neutral. A galvanic cell or electrolytic cell operated most effectively when it is electrically neutral, which means it does not have a net positive or negative charge. The medium 21 could be, but is not limited to, a salt bridge, a semipermeable membrane, or any other conventional semi-porous medium known to the common user. The ions crossing the solution would be the ions present in the blood, which has a high concentration of sodium chloride 24. Other ions are physiologically in the blood including potassium, magnesium, calcium, and others.

The oxidation of glucose takes place via the following simplified reaction:

60₂(g)+Glucose (aq)↔6C0₂(g)+6H₂0(l)ΔG⁰=−2870kJ/mol

However, physiologically, this takes place multiple separate steps in the human body involving electron transfer. This reaction is commonly known as glycolysis and is a ten step reaction involving human enzymes. The net Gibbs free energy value is negative, stating that this reaction is spontaneous at normal human conditions. However, this does not reflect on the kinetics of the reaction, namely that not every step of the ten step reaction is negative, which slows the reaction down. The addition of the power source to this disclosure allows the reaction to proceed at a quicker speed by creating a disequilibrium in the natural reaction, driving the reaction away from the reactants, which are glucose and oxygen, and toward the products, which are carbon dioxide and water. The addition of the six step synthetic reaction also speeds up the reaction by eliminating the positive Gibbs free energy steps of glycolysis. The goal of this disclosure is to push electrons to the cathode faster than they would normally go spontaneously, making the reaction nonspontaneous. The role of pushing makes this now a non-spontaneous reaction which requires energy, which the power source can provide; the now, nonspontaneous reaction, would occur at a quicker speed than the spontaneous reaction would without a power source. As electrons are driven towards the cathode 16, electrons are drawn from the anode 14 to take their place. These electrons would come from glucose in the bloodstream, thus, driving its breakdown into carbon dioxide and water.

The exemplary embodiment has been described with reference to the preferred embodiments. Obviously, modifications and alterations will occur to others upon a reading and understanding of the detailed description. It is intended that the exemplary embodiment be construed as including all such modifications and alterations insofar as they come within the scope of the disclosure and the appended claims.

1. A glucose electrolysis apparatus for reducing osmolality of blood, comprising:
a catheter having:
an anode located at a distal end of said catheter; and
a cathode connected to said anode by a wire located within said catheter;
a mesh covers said anode to exclude molecules from said catheter; and
a power source connected to said wire to drive a reduction reaction forward on the anode surface.

2. The apparatus of claim 1, wherein said anode comprises a bio enzyme cascade.

3. The apparatus of claim 2, wherein said bio enzyme cascade comprises multiple enzymes on a surface of said anode to drive said reduction reaction forward and to oxidize blood glucose to carbon dioxide and water.

4. The apparatus of claim 3, wherein said enzymes comprise one or more of the following:
pyrroloquinoline quinone (PQQ) dependent glucose dehydrogenase, PQQ-dependent 2-gluconate dehydrogenase, aldolase, PQQ-dependent alcohol dehydrogenase, PQQ-dependent aldehyde dehydrogenase, and oxalate oxidase.

5. The apparatus of claim 1, wherein said cathode comprises one or more of the following: silver, silver chloride, platinum, and lithium.

6. The apparatus of claim 1, wherein a conductor is provided to allow a conduit to be completed between said anode and said cathode.

7. The apparatus of claim 6, wherein said conductor comprises one or more of the following: copper, aluminum, silver, steel, iron, gold, or a combination thereof.

8. The apparatus of claim 1, wherein a medium is provided between said anode and said cathode to transfer ions.

9. The apparatus of claim 8, wherein said medium comprises one of a salt bridge, a semi-permeable membrane, and a semi-porous material.

10. The apparatus of claim 1, wherein oxidation of glucose occurs according to a reaction as follows:
60₂(g)+Glucose(aq)↔6C0₂(g)+6H₂0(l)ΔG⁰=−2870kJ/mol

11. A method of oxidation of glucose in blood comprising:
providing a catheter;
providing an anode at a distal end of said catheter;
connecting a cathode to said anode by a reduction wire located within said catheter;
covering said anode with a mesh cover to exclude molecules from entering said catheter;
connecting a power source to said reduction wire to drive a reaction forward on the anode surface; and
inserting said catheter into said bloodstream to break down glucose and reduce osmolality of the blood.

12. The method of claim 11, wherein said anode comprises a bio enzyme cascade.

13. The method of claim 12, wherein said bio enzyme cascade comprises multiple enzymes on a surface of said anode to drive said reduction reaction forward and to oxidize blood glucose to carbon dioxide and water.

14. The method of claim 13, wherein said enzymes comprise one or more of the following:
pyrroloquinoline quinone (PQQ) dependent glucose dehydrogenase, PQQ-dependent 2-gluconate dehydrogenase, aldolase, PQQ-dependent alcohol dehydrogenase, PQQ-dependent aldehyde dehydrogenase, and oxalate oxidase.

15. The method of claim 11, wherein said cathode comprises one or more of the following: silver, silver chloride, platinum, and lithium.

16. The method of claim 11, further including providing a conductor to allow a conduit to be completed between said anode and said cathode.

17. The method of claim 16, wherein said conductor comprises one or more of the following: copper, aluminum, silver, steel, iron, gold, or a combination thereof.

18. The method of claim 11, further including providing a medium between said anode and said cathode to transfer ions.

19. The method of claim 18, wherein said medium comprises one of a salt bridge, a semi-permeable membrane, and a semi-porous material.

20. The method of claim 11, further including oxidation of glucose according to a reaction as follows:
60₂(g)+Glucose(aq)↔6C0₂(g)+6H₂0(l)ΔG⁰=−2870 kJ/mol
